(19) United States
(12) Patent Application Publication
(10) Pub. No.: US 2024/0248097 A1
(43) Pub. Date: Jul. 25, 2024

(54) MASS SPECTROMETRY-BASED CHARACTERIZATION OF ANTIBODIES CO-EXPRESSED IN VIVO

(71) Applicant: Regeneron Pharmaceuticals, Inc., Tarrytown, NY (US)

(72) Inventors: Yuetian Yan, Chappaqua, NY (US); Shunhai Wang, Scarsdale, NY (US)

(21) Appl. No.: 18/422,188

(22) Filed: Jan. 25, 2024

Related U.S. Application Data
(60) Provisional application No. 63/441,050, filed on Jan. 25, 2023.

(51) Int. Cl. G01N 33/68; G01N 30/02; G01N 30/72; G01N 30/96; G01N 33/543

(52) U.S. Cl. CPC G01N 33/6857; G01N 30/7266; G01N 30/96; G01N 33/54306; G01N 2030/027

(57) ABSTRACT

The present inventions generally pertains to methods of characterizing of a protein of interest. In particular, the present inventions pertains to the use of native immunoprecipitation and native strong cation exchange chromatography-mass spectrometry for identifying and quantifying the pairing products of two or more antibodies co-expressed in vivo.

This application claims priority to U.S. Application Ser. No. 63/441,050, filed Jan. 25, 2023, which is hereby incorporated by reference.

### FIELD

This application relates to methods for characterization of therapeutic proteins, such as antibodies and fusion proteins, as well as derivatives and fragments thereof.

### BACKGROUND

Gene therapy that produces therapeutic proteins, for example monoclonal antibodies (mAbs), in vivo, is an attractive alternative to administration of recombinant proteins produced using mammalian host cell lines. In vivo-expressed mAb molecules must be thoroughly characterized for considerations of both drug efficacy and safety. In particular, in cases when multiple antibodies are co-expressed from the same producer cell for combined therapeutic advantages, evaluation and monitoring of correct chain pairing is critical. Such characterization, however, is challenging due to the limitations in sample quantity and matrix complexity.

An antibody typically comprises a first light chain (LC) paired to a first heavy chain (HC) dimerized with a second light chain paired to a second heavy chain. In a monospecific antibody, typically the two heavy chains will be identical to each other and the two light chains will be identical to each other, whereas in a bispecific antibody, typically the two heavy chains will be different from each other and the two light chains will be identical to each other. The specific combination of light chain and heavy chain pairs, referred to herein as chain pairing, determines the structure and function of a particular antibody or antibody-derived protein. When two or more antibodies are co-expressed in the same cell, the various light chains and heavy chains may pair indiscriminately, forming a mix of correctly paired and mispaired protein products. For example, the co-expression of two mAbs with different heavy chain and light chain sequences can result in the production of up to 10 different HC-LC pairs, two of which represent the desired mAbs and eight of which represent mispaired impurities.

Electrospray ionization mass spectrometry (ESI MS)-based intact protein analysis has become an essential tool for the characterization of therapeutic proteins during development. Most commonly, mass spectrometry (MS) is coupled with reversed phase liquid chromatography (RPLC) under denaturing conditions. However, the sensitivity of this method, and the signal-to-noise ratio produced by the resulting complex sample with a wide range of analyte charge states, has limits which may make it unreliable for accurate quantitation of low-abundance antibodies.

The use of native strong cation exchange chromatography (SCX)-MS provides a number of advantages for analysis of therapeutic proteins compared to conventional denaturing RPLC-MS. Native SCX-MS may demonstrate high sensitivity and a wide dynamic range compared to RPLC, and a superior ability to separate a target analyte from matrix, such as, for example, serum proteins in a serum sample.

Therefore, there exists a need for sensitive methods to characterize therapeutic proteins and peptides, such as therapeutic antibodies, in a sample comprising a complex matrix. The inventions provide new native immunoprecipitation and native LC-MS methods for characterizing a therapeutic protein, suitable for development of therapeutic antibodies. For example, using native immunoprecipitation followed by native LC-MS and bottom-up approaches, novel methods were developed for successful characterization of chain pairing and N-linked glycosylation of a two-mAb cocktail co-expressed using different in vivo gene delivery platforms in mice.

### SUMMARY

A method has been developed for characterizing chain pairing of an antibody cocktail expressed in vivo. For example, a sample including an antibody cocktail of two or more antibodies co-expressed in vivo may be subjected to immunoprecipitation under native or near-native conditions. The immobilized antibodies may be contacted with a digestive enzyme, for example IdeS or a variant thereof, to produce antibody fragments, for example Fab₂ fragments. The antibody fragments may then be eluted and subjected to native strong cation exchange chromatography-mass spectrometry (nSCX-MS) analysis to identify and quantify correctly paired and mispaired antibody products.

A method has also been developed for characterizing the glycosylation profile of an antibody cocktail expressed in vivo. For example, a sample including an antibody cocktail of two or more antibodies co-expressed in vivo may be subject to immunoprecipitation under native or near-native conditions. The immobilized antibodies may be contacted with a digestive enzyme, for example IdeS or a variant thereof, to produce antibody fragments, for example Fc fragments. The antibody fragments may then be eluted and contacted to another digestive enzyme, for example trypsin, to produce a peptide digest. The peptide digest may be subjected to reversed phase liquid chromatography-mass spectrometry (RPLC-MS) analysis to characterize the glycosylation profile of the antibodies making up the antibody cocktail.

The inventions provide methods for characterizing assembly of subunits of at least one multi-subunit protein of interest. The methods can comprise: (a) contacting a sample including at least one multi-subunit protein of interest to a solid-phase substrate under native or near-native conditions, wherein the solid-phase substrate comprises a capture antibody that binds to at least one subunit of the at least one multi-subunit protein of interest, to form immobilized proteins; (b) eluting the immobilized proteins to form an enriched sample; and (c) subjecting the enriched sample to liquid chromatography-mass spectrometry (LC-MS) analysis under native conditions to characterize assembly of subunits of the at least one multi-subunit protein of interest.

In an aspect, the at least one multi-subunit protein is selected from a group consisting of an antibody, a monoclonal antibody, a bispecific antibody, an antibody fragment, an antibody-derived protein, an antigen-binding protein, an antibody-drug conjugate, or a fusion protein.

In an aspect, the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

The inventions also provide methods for identifying and/or quantifying correctly paired antibodies from a sample comprising two or more antibodies co-expressed in vivo. The methods can comprise: (a) contacting a sample including two or more antibodies co-expressed in vivo to a solid-phase substrate under native or near-native conditions to form immobilized antibodies; (b) contacting the immobilized antibodies to a digestive enzyme to produce unbound fragments of the antibodies; (c) eluting the unbound fragments; and (d) subjecting the eluted fragments to native strong cation exchange chromatography-mass spectrometry (nSCX-MS) analysis to identify and/or quantify correctly paired antibodies.

In an aspect, the solid-phase substrate is selected from a group consisting of a microplate, resin, and beads. In an aspect, the solid-phase substrate comprises beads. In a specific aspect, the beads are agarose beads or magnetic beads.

In an aspect, the binding is performed by an antibody-binding molecule adhered to the solid-phase substrate. In a specific aspect, the antibody-binding molecule is Protein A, Protein G, or an anti-Fc antibody. In an aspect, the binding is performed by an antibody adhered to the solid-phase substrate.

In an aspect, the digestive enzyme is selected from a group consisting of pepsin, trypsin, Tryp-N, chymotrypsin, Lys-N, Lys-C, Asp-N, Arg-C, Glu-C, papain, IdeS, or a variant thereof.

In an aspect, the unbound fragments are Fab fragments, Fab′ fragments, Fab₂ fragments, F(ab′)₂ fragments, Fc fragments, Fv fragments, Fd fragments, or Fd′ fragments.

The inventions further provide methods for identifying and/or quantifying correctly paired antibodies from a sample comprising two or more antibodies co-expressed in vivo. The methods can comprise: (a) contacting a sample including two or more antibodies co-expressed in vivo to a solid-phase substrate under native or near-native conditions, wherein the solid-phase substrate comprises anti-Fc antibodies, to form immobilized antibodies; (b) contacting the immobilized antibodies to digestive conditions including IdeS or a variant thereof to form free Fab₂ fragments; (c) eluting the free Fab₂ fragments to form eluted Fab₂ fragments; and (d) subjecting the eluted Fab₂ fragments to native strong cation exchange chromatography-mass spectrometry (nSCX-MS) analysis to identify and/or quantify correctly paired antibodies.

In an aspect, the two or more antibodies are selected from a group consisting of therapeutic antibodies, monoclonal antibodies, bispecific antibodies, multispecific antibodies, antibody-drug conjugates, antibody fusion proteins, or antibody fragments.

In an aspect, the two or more antibodies are casirivimab and imdevimab.

In an aspect, the sample is a biological sample. In a specific aspect, the biological sample is selected from a group consisting of whole blood, plasma, serum, saliva, or organ tissue.

In an aspect, the sample is serum.

In an aspect, at least one subunit from each of the at least two antibodies is similar in mass. In a specific aspect, the at least one subunit from each of the at least two antibodies has a difference in mass of less than 10 daltons, of less than 9 daltons, of less than 8 daltons, of less than 7 daltons, of less than 6 daltons, of less than 5 daltons, of less than 4 daltons, of less than 3 daltons, of less than 2 daltons, or of less than 1 dalton.

In an aspect, the solid-phase substrate is selected from a group consisting of a microplate, resin, agarose beads, and magnetic beads.

In an aspect, the solid-phase substrate comprises agarose beads.

In an aspect, the method further comprises a step of washing the solid-phase substrate after contacting the sample to the solid-phase substrate.

In an aspect, the eluting comprises a step of centrifuging the solid-phase substrate and the free Fab₂ fragments.

In an aspect, the mass spectrometer is an electrospray ionization mass spectrometer, nano-electrospray ionization mass spectrometer, or a triple quadrupole mass spectrometer.

The inventions additionally provide methods for characterizing at least one antibody of interest expressed in vivo. The methods can comprise: (a) contacting a sample including at least one antibody of interest expressed in vivo to a solid-phase substrate under native or near-native conditions, wherein the solid-phase substrate comprises anti-Fc antibodies, to form immobilized antibodies; (b) contacting the immobilized antibodies to digestive conditions including IdeS or a variant thereof to form free Fab₂ fragments; (c) eluting the free Fab₂ fragments to form eluted Fab₂ fragments; and (d) subjecting the eluted Fab₂ fragments to native size exclusion chromatography-mass spectrometry (nSEC-MS) or native strong cation exchange chromatography-mass spectrometry (nSCX-MS) analysis to characterize the at least one antibody of interest.

The inventions also provide methods for identifying and/or quantifying impurities in the production of a bispecific antibody. The methods can comprise: (a) contacting a sample including a bispecific antibody and at least one impurity to a solid-phase substrate under native or near-native conditions, wherein the solid-phase substrate comprises anti-Fc antibodies, to form immobilized antibodies; (b) contacting the immobilized antibodies to digestive conditions including IdeS or a variant thereof to form free Fab₂ fragments; (c) eluting the free Fab₂ fragments to form eluted Fab₂ fragments; and (d) subjecting the eluted Fab₂ fragments to nSCX-MS analysis to identify and/or quantify the impurities.

In an aspect, the at least one impurity is a side product impurity. In a specific aspect, the at least one side product impurity is a monospecific antibody.

In an aspect, the bispecific antibody is produced by expression of two distinct heavy chains and one common light chain. In a specific aspect, the at least one impurity is a result of homodimerization of one of the heavy chains.

The inventions further provide methods for characterizing a glycosylation profile of a protein of interest. The methods can comprise: (a) contacting a sample including a protein of interest to a solid-phase substrate under native or near-native conditions, wherein the solid-phase substrate comprises a capture antibody that binds to the protein of interest, to form immobilized proteins; (b) eluting the immobilized proteins to form an enriched sample; and (c) subjecting the enriched sample to native liquid chromatography-mass spectrometry analysis to characterize a glycosylation profile of the protein of interest.

The inventions additionally provide methods for characterizing glycosylation profiles of antibodies from a sample including at least two antibodies co-expressed in vivo. The methods can comprise: (a) contacting a sample including at least two antibodies co-expressed in vivo to a solid-phase substrate under native or near-native conditions, wherein the solid-phase substrate comprises anti-Fc antibodies, to form immobilized antibodies; (b) contacting the immobilized antibodies to digestive conditions including IdeS or a variant thereof to form immobilized Fc fragments; (c) eluting the immobilized Fc fragments to form eluted Fc fragments; (d) subjecting the eluted Fc fragments to digestive conditions to form a peptide digest; and (e) subjecting the peptide digest to reversed phase liquid chromatography-mass spectrometry (RPLC-MS) analysis to characterize glycosylation profiles of the antibodies.

The inventions also provide methods for selecting an expression platform for producing at least one protein of interest. The methods can comprise: (a) obtaining a sample including at least one protein of interest produced from a first expression platform; (b) contacting the sample to a solid-phase substrate under native or near-native conditions, wherein the solid-phase substrate comprises a capture antibody that binds to the at least one protein of interest, to form immobilized proteins; (c) eluting the immobilized proteins to form an enriched sample; (d) subjecting the enriched sample to nLC-MS analysis to characterize the at least one protein of interest produced from the expression platform; (e) repeating steps (a)-(d) using at least one alternative expression platform; (f) comparing the results of step (d) for each of the first and the at least one alternative expression platforms; and (g) selecting an expression platform on the basis of the comparison.

In an aspect, the first expression platform and the at least one alternative expression platform comprise recombinant expression platforms and/or gene therapy expression platforms.

The inventions further provide methods for selecting a gene therapy platform for producing an antibody cocktail. The methods can comprise: (a) obtaining a sample including an antibody cocktail produced from a first gene therapy platform; (b) contacting the sample to a solid-phase substrate under native or near-native conditions, wherein the solid-phase substrate comprises anti-Fc antibodies, to form immobilized antibodies; (c) contacting the immobilized antibodies to digestive conditions to form free Fab₂ fragments; (d) eluting the free Fab₂ fragments to form eluted Fab₂ fragments; (e) subjecting the eluted Fab₂ fragments to nSCX-MS analysis to characterize the correct subunit pairing of the antibody cocktail produced from the gene therapy platform; (f) repeating steps (a)-(e) using at least one alternative gene therapy platform; (g) comparing the results of step (e) for each of the first and the at least one alternative gene therapy platforms; and (h) selecting a gene therapy platform on the basis of the comparison.

The inventions additionally provide methods for selecting a protein of interest. The methods can comprise: (a) contacting a sample including a first protein of interest to a solid-phase substrate under native or near-native conditions, wherein the solid-phase substrate comprises a capture antibody that binds to the protein of interest, to form immobilized proteins; (b) eluting the immobilized proteins to form an enriched sample; (c) subjecting the enriched sample to nLC-MS analysis to characterize the protein of interest; (d) repeating steps (a)-(c) using at least one alternative protein of interest; (e) comparing the results of step (c) for each of the first and the at least one alternative proteins of interest; and (f) selecting a protein of interest on the basis of the comparison.

The inventions also provide methods for selecting an antibody cocktail. The methods can comprise: (a) contacting a sample including a first antibody cocktail to a solid-phase substrate under native or near-native conditions, wherein the solid-phase substrate comprises an anti-Fc antibody, to form immobilized antibodies; (b) contacting the immobilized antibodies to digestive conditions to form free Fab₂ fragments; (c) eluting the free Fab₂ fragments to form eluted Fab₂ fragments; (d) subjecting the eluted Fab₂ fragments to nSCX-MS analysis to characterize the correct subunit pairing of the antibody cocktail; (e) repeating steps (a)-(d) using at least one alternative antibody cocktail; (f) comparing the results of step (d) for each of the first and the at least one alternative antibody cocktails; and (g) selecting an antibody cocktail on the basis of the comparison.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product that is shown that may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to an reference product already approved in Japan. In India, biosimilars currently are referred to as “similar biologics,” and refer to a similar biologic product is that which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production and analysis of biosimilars and its synonyms under these and any revised definitions can be undertaken according to the inventions.

These, and other, aspects of the inventions will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the inventions.

### BRIEF DESCRIPTION OF THE DRAWINGS

FIG. 1 illustrates a workflow of a native immunoprecipitation and native liquid chromatography-mass spectrometry method for characterizing at least one protein of interest.

FIG. 2A shows size exclusion chromatography (SEC)-MS chromatograms and mass spectra for mAbs produced using three gene therapy platforms.

FIG. 2B shows the masses of Fab₂ fragments produced using three gene therapy platforms analyzed by SEC-MS.

FIG. 3A shows the theoretical masses of Fab₂ fragments produced by co-expressing mAb1 and mAb2.

FIG. 3B shows SEC-MS chromatograms and mass spectra for Fab₂ fragments from mAb1 and mAb2 cocktail produced using three gene therapy platforms.

FIG. 4 shows total ion chromatograms from strong cation exchange chromatography (SCX)-MS analysis of mAb1 or mAb2 Fab fragments produced using a gene therapy platform (panel 1 and 2); and total ion chromatograms (native, panel 7) and extracted ion chromatograms (with post-column denaturation, panel 3-6) from strong cation exchange chromatography (SCX)-MS analysis of co-expressed mAb1 and mAb2 cocktail Fab fragments produced using a gene therapy platform.

FIG. 5A shows SCX-MS analysis of mAb1 and mAb2 cocktail Fab fragments produced using three gene therapy platforms.

FIG. 5B shows a quantitation of SCX-MS analysis of mAb1 and mAb2 cocktail Fab fragments produced using three gene therapy platforms.

FIG. 6 shows a predicted SCX elution order of mAb1 and mAb2 cocktail Fab₂ fragments.

FIG. 7A shows SCX-MS analysis of singly expressed mAb1, singly expressed mAb2, and co-expressed mAb1/mAb2 cocktail Fab₂ fragments produced using gene therapy platform I.

FIG. 7B shows deconvoluted mass spectra from SCX-MS analysis of co-expressed mAb1 and mAb2 cocktail Fab₂ fragments produced using gene therapy platform I.

FIG. 7C shows SCX-MS analysis of mAb1 and mAb2 cocktail Fab₂ fragments produced using gene therapy platform II.

FIG. 7D shows deconvoluted mass spectra from SCX-MS analysis of mAb1 and mAb2 cocktail Fab₂ fragments produced using gene therapy platform II.

FIG. 7E shows SCX-MS analysis of mAb1 and mAb2 cocktail Fab₂ fragments produced using gene therapy platform III.

FIG. 7F shows deconvoluted mass spectra from SCX-MS analysis of mAb1 and mAb2 cocktail Fab₂ fragments produced using gene therapy platform III.

FIG. 8A shows a MS-based quantitation of SCX-MS analysis of mAb1 and mAb2 cocktail Fab₂ fragments produced using three gene therapy platforms.

FIG. 8B shows a distribution of correctly and incorrectly paired products identified using SCX-MS analysis of mAb1 and mAb2 cocktail Fab₂ fragments.

FIG. 9 shows a workflow of a native immunoprecipitation and native liquid chromatography-mass spectrometry method for glycopeptide analysis.

FIG. 10 shows intact mass analysis of mAb2 Fc fragments produced using three gene therapy platforms.

FIG. 11A shows a workflow of bottom-up glycopeptide analysis.

FIG. 11B shows a bottom-up glycopeptide analysis of mAb1 and mAb2 cocktail Fc fragments produced using gene therapy platform I.

FIG. 11C shows a bottom-up glycopeptide analysis of mAb1 and mAb2 cocktail Fc fragments produced using gene therapy platform II.

FIG. 11D shows a bottom-up glycopeptide analysis of mAb1 and mAb2 cocktail Fc fragments produced using gene therapy platform III.

FIG. 12 shows a comparison of glycoforms identified using intact mass analysis (black) and bottom-up glycopeptide analysis (black and red).

### DETAILED DESCRIPTION

Gene therapy that produces therapeutic proteins, for example monoclonal antibodies (mAbs), in vivo, is an attractive alternative to administration of recombinant protein produced using mammalian host cell lines. Similar to recombinant mAbs, in vivo-expressed mAb molecules must be thoroughly characterized (for example, primary sequence, post-translational modifications, and tertiary structure) for considerations of both drug efficacy and safety. In particular, in cases when multiple antibodies are co-expressed from the same producer cell for combined therapeutic advantages, evaluation and monitoring of correct chain pairing is critical. Such characterization, however, is challenging due to the limitations in sample quantity and matrix complexity.

REGEN-COV (casirivimab and imdevimab) is an investigational antibody cocktail therapy developed by Regeneron Pharmaceuticals, Inc. for the treatment of coronavirus disease 2019 (COVID-19) caused by severe acute respiratory syndrome coronavirus 2 (SARS-COV-2) (Hansen et al., 2020, Science, 369:1010-1014; Baum et al., 2020, Science, 370:1110-1115; Weinreich et al., 2021, N Engl J Med, 384:238-251). The antibody cocktail includes two humanized IgG1 monoclonal antibodies (herein referred to as mAb1 and mAb2), which are designed to target non-overlapping epitopes on the SARS-COV-2 spike protein, and thereby blocking the interaction of SARS-COV-2 virus with human ACE2, and preventing viral escape due to rapid genetic mutation of the virus (Hansen et al.; Baum et al., 2020, Science, 369:1014-1018).

When co-expressing two mAbs with different heavy chain (HC) and light chain (LC) sequences, up to 10 different HC-LC pairs may be produced. Separating the correctly paired mAbs and characterizing the mispairing of the products presents many challenges due to potentially similar sizes, charges, structures, and sequences between different analytes. This challenge is greatly increased when the products are in a sample with a complex matrix, such as serum.

Relatedly, the production of bispecific antibody (bsAb) products involves producing two different HC sequences, and either one or two LC sequences. This production can result in either 3 or 10 different HC-LC pairs, depending on the number of LC sequences. Separation and characterization of these products presents a significant challenge due to their overall similarity.

Another important consideration regarding the efficacy of therapeutic proteins is variation in protein glycosylation profiles. Human IgG subclasses share a conserved amino acid sequence in the Fc region, including a single N-glycosylation site (N297). Fc glycosylation is crucial to the interaction between Fc and different types of receptors, and therefore related to the biophysical profile of an IgG molecule, such as lifetime and effector functions. For instance, antibodies with Fc-afucosylation exhibit stronger binding affinity to Fcγ receptor III and enhanced antibody-dependent cellular cytotoxicity (ADCC); Fc-galactosylation is associated with a strong binding affinity to C1q and enhanced complement-dependent cytotoxicity (CDC); and antibodies with N-linked mannose-5 glycan (Man5) may display higher immunogenicity and faster clearance in serum. Therefore, characterizing the glycan profile of a potential therapeutic protein, such as Fc glycosylation of a therapeutic antibody, is an important aspect of drug development, and methods are needed for effectively doing so in complex samples.

Electrospray ionization mass spectrometry (ESI MS)-based intact protein analysis has become an essential tool for the characterization of therapeutic proteins during development. Most commonly, MS is coupled with reversed phase liquid chromatography (RPLC) under denaturing conditions. However, the sensitivity of this method, and the signal-to-noise ratio produced by the resulting complex sample with a wide range of analyte charge states, has limits which may make it unreliable for accurate quantitation of low-abundance antibodies.

Recently, LC-MS systems comprising native ion exchange chromatography coupled online to ESI MS have been described (Yan et al., 2020, J Am Soc Mass Spectrom, 31:2171-2179). The use of native strong cation exchange chromatography (SCX)-MS provides a number of advantages for analysis of therapeutic proteins compared to conventional denaturing RPLC-MS. Native SCX-MS may demonstrate high sensitivity and a wide dynamic range compared to RPLC, and a superior ability to separate a target analyte from matrix, such as, for example, serum proteins in a serum sample. A native SCX-MS profile may also feature superior MS spatial resolution, making it easier to detect protein variants or biotransformation products.

As described above, there exists a need for sensitive methods to characterize therapeutic proteins and peptides, such as therapeutic antibodies, in a sample. The inventions provide novel native immunoprecipitation and native LC-MS methods for characterizing a therapeutic protein, suitable for development of therapeutic antibodies. Using native immunoprecipitation followed by native MS and bottom-up approaches, inventive methods were developed for successful characterization of chain pairing and N-linked glycosylation of a two-mAb cocktail co-expressed using different in vivo gene delivery platforms in mice.

Unless described otherwise, all technical and scientific terms used herein have the same meaning as commonly understood by one of ordinary skill in the art to which these inventions belongs. Although any methods and materials similar or equivalent to those described herein can be used in the practice or testing, particular methods and materials are now described.

The term “a” should be understood to mean “at least one”. As used herein, the terms “include,” “includes,” and “including” are meant to be non-limiting and are understood to mean “comprise,” “comprises,” and “comprising” respectively. The word “said” can be used in the same manner as “the”.

The term “about” in the context of numerical values and ranges refers to values or ranges that approximate or are close to the recited values or ranges such that the inventions can perform, such as having a desired rate, amount, density, degree, increase, decrease, percentage, ratio, value, purity, pH, concentration, presence of a form or variant, temperature or amount of time, as is apparent from the teachings contained herein. The term permits standard variation as would be understood by those of ordinary skill in the art, and where ranges are provided, endpoints are included. For example, “about” can signify values either above or below the stated value in a range of approximately +/−10% or more or less depending on the ability to perform. Thus, this term encompasses values beyond those simply resulting from systematic error.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012). Proteins can comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

For example, the protein of interest can be a recombinant protein, an in vivo product of gene therapy, a therapeutic protein, an antibody, a bispecific antibody, a multispecific antibody, antibody fragment, monoclonal antibody, antigen-binding protein, fusion protein, scFv, a multi-subunit protein, a receptor, a receptor ligand, and combinations thereof.

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

“Fc-fusion proteins” are a type of fusion protein and comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci. USA 88: 10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which can comprise a hinge region followed by a CH2 and CH3 domain of an immunoglobulin. The Fc-fusion protein also can contain two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-IR1 extracellular region fused to Fc of hIgGl; see U.S. Pat. No. 6,927,044, or a VEGF Trap (for example, Aflibercept, which contains the Ig domain 2 of the VEGF receptor Fltl fused to the Ig domain 3 of the VEGF receptor Flkl fused to Fc of hIgG1 See U.S. Pat. Nos. 7,087,411 and 7,279,159.

As used herein, the term “therapeutic protein” refers to any protein that can be administered to a subject for the treatment of a disease or disorder. Therapeutic proteins can be used in, but are not limited to, the production of biological and pharmaceutical products. Protein-based therapeutics can have any amino acid sequence, and include any protein, polypeptide, or peptide that is desired to be manufactured. Included are, but not limited to, viral proteins, bacterial proteins, fungal proteins, plant proteins and animal (including human) proteins. Protein types can include, but are not limited to, antibodies, receptors, Fc-containing proteins, trap proteins, enzymes, factors, repressors, activators, ligands, reporter proteins, selection proteins, protein hormones, protein toxins, structural proteins, storage proteins, transport proteins, neurotransmitters and contractile proteins. Derivatives, components, chains and fragments of the above also are included. The sequences can be natural, semi-synthetic or synthetic. Proteins of interest and polypeptides of interest are encoded by “genes of interest,” which also can be referred to as “polynucleotides of interest.”

A therapeutic protein may be any protein with a pharmacological effect, for example, an antibody, a soluble receptor, an antibody-drug conjugate, an antigen-binding protein, or an enzyme. The therapeutic protein can be a monoclonal antibody. Two or more therapeutic proteins may be present in the same sample. Two or more therapeutic proteins may be co-expressed in a cell. The therapeutic protein can be an anti-SARS-COV-2 antibody, including casirivimab or imdevimab. Multiple therapeutic proteins may be co-administered in order to achieve a pharmacological effect, for example, to prevent viral escape due to mutation of a target virus. As used herein, the term “antibody cocktail” refers to co-administered therapeutic proteins comprising at least two therapeutic antibodies. An antibody cocktail can comprise REGEN-COV.

Therapeutic proteins include, but are not limited to, a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab fragment or a F(ab′)₂ fragment, an IgD antibody, an IgE antibody, an IgM antibody, an IgG antibody, an IgG1 antibody, an IgG2 antibody, an IgG3 antibody, or an IgG4 antibody. Chimeric antibodies, such as a IgG2/IgG4 antibody, a IgG2/IgG1 antibody, and a IgG2/IgG1/IgG4 antibody, also can be used.

The antibody can selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-Dll4 antibody, an anti-Angiopoetin-2 antibody (for example an anti-ANG2 antibody as described in U.S. Pat. No. 9,402,898), an anti-Angiopoetin-Like 3 antibody (for example an anti-AngPtl3 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-C5 antibody as described in U.S. Pat. Appln. Pub. No US2015/0313194A1), an anti-TNF antibody, an anti-epidermal growth factor receptor antibody (for example an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat. No. 8,871,209 or 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-ILIR antibody, an interleukin 4 receptor antibody (e.g an anti-IL4R antibody as described in U.S. Pat. Appln. Pub. No. US2014/0271681A1 or U.S. Pat. No. 8,735,095 or 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088295A1 and US20150266966A1, and in U.S. Application No. 62/222,605), an anti-Cluster of differentiation 20 (for example an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation 48 (for example anti-CD48 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Activin A antibody. The bispecific antibody can be selected from the group consisting of an anti-CD3 x anti-CD20 bispecific antibody (as described in U.S. Pat. Appln. Pub. Nos. US2014/0088295A1 and US20150266966A1), an anti-CD3 x anti-Mucin 16 bispecific antibody (for example, an anti-CD3 x anti-Muc16 bispecific antibody), and an anti-CD3 x anti-Prostate-specific membrane antigen bispecific antibody (for example, an anti-CD3 x anti-PSMA bispecific antibody). See also U.S. Patent Publication No. US 2019/0285580 A1. Also included are a Met x Met antibody, an agonist antibody to NPR1, an LEPR agonist antibody, a BCMA x CD3 antibody, a MUC16 x CD28 antibody, a GITR antibody, an IL-2Rg antibody, an EGFR x CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured according to the inventions. Exemplary antibodies include Alirocumab, Atoltivimab, Maftivimab, Odesivimab, Odesivivmab-ebgn, Casirivimab, Imdevimab, Cemiplimab and Cemiplimab-rwlc (human IgG4 monoclonal antibody that binds PD-1), Dupilumab (human monoclonal antibody of the IgG4 subclass that binds to the IL-4R alpha (a) subunit and thereby inhibits Interleukin 4 (IL-4) and Interleukin 13 (IL-13) signalling), Evinacumab, Evinacumab-dgnb, Fasinumab, Fianlimab, Garetosmab, Itepekimab Nesvacumab, Odrononextamab, Pozelimab, Sarilumab, Trevogrumab, and Rinucumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

The inventions also are amenable to other therapeutic proteins, including fusion proteins. Fusion proteins include Fc-Fusion proteins, such as Receptor-Fc-fusion proteins, for example Trap proteins. The protein of interest can be a recombinant protein that contains an Fc moiety and another domain, (for example, an Fc-fusion protein). The Fc-fusion protein can be a receptor Fc-fusion protein, which contains one or more extracellular domain(s) of a receptor coupled to an Fc moiety. The Fc moiety can comprise a hinge region followed by a CH2 and CH3 domain of an IgG. The receptor Fc-fusion protein can contain two or more distinct receptor chains that bind to either a single ligand or multiple ligands. For example, an Fc-fusion protein is a TRAP protein, such as for example an IL-1 trap (for example, rilonacept, which contains the IL-IRAcP ligand binding region fused to the Il-1R1 extracellular region fused to Fc of hIgG1; see U.S. Pat. No. 6,927,044, or a VEGF trap (for example, aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). The Fc-fusion protein can be a ScFv-Fc-fusion protein, which contains one or more of one or more antigen binding domain(s), such as a variable heavy chain fragment and a variable light chain fragment, of an antibody coupled to an Fc moiety. Derivatives, components, domains, chains and fragments of the above also are included.

Other proteins lacking Fc portions, such as recombinantly produced enzymes and mini-traps, also can be made according to the inventions. Mini-traps are trap proteins that use a multimerizing component (MC) instead of an Fc portion, and are disclosed in U.S. Pat. Nos. 7,279,159 and 7,087,411. Derivatives, components, domains, chains and fragments of the above also are included.

As used herein, the term “recombinant protein” refers to a protein produced as the result of the transcription and translation of a gene carried on a recombinant expression vector that has been introduced into a suitable host cell. The recombinant protein can be an antibody, for example, a chimeric, humanized, or fully human antibody. The recombinant protein can be an antibody of an isotype selected from group consisting of: IgG, IgM, IgA1, IgA2, IgD, or IgE. The antibody molecule can be a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FRI, CDR1, FR2, CDR2, FR3, CDR3, and FR4. An amino acid consensus sequence may be defined based on a side-by-side analysis of two or more CDRs. The term “antibody,” as used herein, also includes antigen-binding fragments of full antibody molecules.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)₂ fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment can comprise a sufficient amino acid sequence of the parent antibody of which it is a fragment that it binds to the same antigen as does the parent antibody; and a fragment can bind to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KA-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22343 filed Jul. 29, 2016; and U.S. Ser. No. 15/814,095, filed Nov. 15, 2017.

A bispecific antibody can be produced by expressing in a cell (e.g., recombinantly in vitro or through gene therapy in vivo) both a first heavy chain specific to a first epitope, and a second heavy chain specific to a second epitope. Since the antibody contains two heavy chains, at least three forms of antibody would be produced by the cell: a homodimer specific to the first epitope having two identical first heavy chains (a.k.a. homo-B), a homodimer specific to the second epitope having two identical second heavy chains (a.k.a. homo-A), and a heterodimer specific to both epitopes and having both a first and a second heavy chain (a.k.a. hetero-AB). In some cases, the biophysical attributes of the homodimers and the heterodimer (e.g., mass, isoelectric point, amino acid content, and the like) are similar enough to make specific identification and quantification of each species difficult. The inventions provide methods for characterizing correctly paired and mispaired products from a cell producing a bispecific antibody.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present inventions can be prepared using a wide variety of techniques known in the art including the use of hybridoma, recombinant, phage display technologies, gene therapy, or a combination thereof.

As used herein, the term “multimer” and the phrases “multimeric protein” and “multisubunit protein” are used interchangeably to denote a protein made of more than one component subunit. The subunits may be bound together or otherwise associated to form the multimer. The binding or association may be via any one or more intermolecular bonds, including covalent and non-covalent bonds. A “homodimer” is a multimer comprising two or more subunits that are the same or functionally equivalent. As used herein, a homodimer comprises at least two polypeptide chains that are the same or functionally equivalent, but the homodimer may include additional subunits as well. For example, a monoclonal antibody contains two identical heavy chains. As such, the monoclonal antibody may be considered to be a “homodimer”. However, a complete canonical monoclonal antibody also contains two light chains and thus can be referred to as a tetramer. A “heterodimer” is a multimer comprising two or more subunits that are not the same or are not functionally equivalent. The heterodimer may contain additional subunits beside the two dissimilar subunits. For example, a bispecific antibody contains two heavy chains and two light chains, such that one half of the antibody (e.g., one heavy chain and one light chain) binds one epitope and the other half of the antibody (e.g., another heavy chain and the same light chain, the same heavy chain and another light chain, or another light chain and another heavy chain) specifically binds to another epitope. The bispecific antibody is a tetramer. In some cases, the bispecific antibody is referred to as a heterodimer, as that term relates to the heavy chains not being the same or not being functionally equivalent.

As used herein, the term “subunit” or “component subunit” means a component of a multimer, usually (but not always) a polypeptide. The component polypeptide is a single chain and can be of any size from three amino acids to several thousands of amino acids long.

The protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

The sample including the protein of interest can be prepared prior to or following enrichment steps, separation steps, and/or analysis steps. Preparation steps can include alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. A non-reduced preparation may be used, for example, in order to preserve an endogenous disulfide bond between Fab arms of an antibody or antibody-derived protein. A partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

IdeS or a variant thereof can be used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, the terms “oxidative species,” “OS,” or “oxidation variant” refer to the variants of a protein formed by oxidation. Oxidation variants can result from oxidation occurring at histidine, cysteine, methionine, tryptophan, phenylalanine and/or tyrosine residues.

As used herein, a “sample” can be obtained from any step of the bioprocess, such as cell culture fluid (CCF), harvested cell culture fluid (HCCF), any step in the downstream processing, drug substance (DS), or a drug product (DP) comprising the final formulated product. The sample can be selected from any step of the downstream process of clarification, chromatographic production, viral inactivation, or filtration. The drug product can be selected from manufactured drug product in the clinic, shipping, storage, or handling.

The sample is a biological sample. As used herein, the term “biological sample” refers to a sample taken from a living organism, for example a human or non-human mammal. A biological sample may comprise or consist of, for example, whole blood, plasma, serum, saliva, tears, semen, cheek tissue, organ tissue, urine, feces, skin, or hair. A sample may be taken from a patient, for example, a clinical sample. A sample may be taken from a non-human animal, for example, a preclinical sample. A sample may be taken from a non-human animal subjected to gene therapy in order to produce at least one protein of interest that may be included in the sample. A sample is a further processed form of any of the aforementioned examples of samples.

As used herein, the term “impurity” can include any undesirable protein present in a protein sample or protein biopharmaceutical product. Impurity can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S—S linked, oxidized, or altered conjugated forms (e.g., glycosylation, phosphorylation). Modified forms can also include any post-translational modification form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

In the production of multisubunit proteins such as antibodies, mispairing between subunits can form alternative antibodies that may be considered impurities in the sample. For example, co-expression of two or more monospecific antibodies in a cell may result in mispairing between different heavy chains and light chains, resulting in the production of incorrectly paired antibody impurities. Expression of a bispecific antibody comprising two different heavy chains may result in the production of incorrectly paired monospecific homodimer impurities. Impurities comprising the subunits of the desired multisubunit protein may be particularly challenging to separate from the desired protein due to their similar size, charge, sequence and/or structure.

The method for characterizing and/or quantifying a protein of interest can optionally comprise enriching a protein of interest in the sample matrix using immunoprecipitation (IP). As used herein, the term “immunoprecipitation” can include a process of precipitating a protein antigen out of solution using an antibody that specifically binds to that particular protein. Immunoprecipitation may be direct, in which antibodies for the target protein are immobilized on a solid-phase substrate, or indirect, in which free antibodies are added to the protein mixture and later captured with, for example, protein A/G beads. IP may be conducted under native or near-native conditions, such that the native structure of a protein or proteins of interest are substantially preserved; for example, heavy chain and light chain pairing of an antibody or antibody-derived protein of interest.

The solid-phase substrate may be beads, for example agarose beads or magnetic beads. Beads may be coated in streptavidin in order to facilitate adherence to an antibody. A biotinylated “capture” antibody may then be contacted to the streptavidin-coated beads, adhering to the beads and forming “immunoprecipitation beads” capable of binding to the antigen of the adhered antibody. The adhered capture antibody may be an anti-Fc antibody, and may specifically be an anti-human Fc antibody.

An anti-human Fc antibody will preferentially bind to the Fc domain of any human antibody, such as, for example, a therapeutic antibody, and thus may be used to immunoprecipitate or “pull down” a therapeutic antibody from a sample, allowing it to be enriched for analysis. After immunoprecipitation of a therapeutic antibody, a digestive enzyme may be contacted to the immunoprecipitation mixture to cleave the therapeutic antibody and release antibody fragments that may then be eluted for further analysis. For example, IdeS or variants thereof are used as a digestive enzyme. IdeS cleavage produces two antibody fragments: an Fc fragment and a Fab₂ fragment. When the Fc domain of a therapeutic antibody is bound to an anti-human Fc capture antibody, cleavage with IdeS will result in the release of an unbound Fab₂ fragment, which can then be eluted for further analysis. For example, eluted Fab₂ fragments are subjected to liquid chromatography-mass spectrometry analysis. The LC-MS analysis can be native SEC-MS or native SCX-MS. Alternatively, or additionally, Fc domains bound to an anti-human Fc capture antibody may be eluted for further analysis. For example, eluted Fc fragments are subjected to liquid chromatography-mass spectrometry analysis. The LC-MS analysis can be native SEC-MS or native SCX-MS.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC).

Methods for characterizing and/or quantifying a protein of interest can include the use of strong cation exchange (SCX) chromatography. Cation exchange chromatography is a subset of ion exchange chromatography that uses a stationary phase presenting a negatively charged functional group in order to capture positively charged analytes. The pH of the chromatography buffer can be gradually adjusted in order to release and elute the analytes in order of pI.

Cation exchange chromatography uses a “cation exchange chromatography material.” Cation exchange chromatography can be further subdivided into, for example, strong cation exchange (SCX) or weak cation exchange, depending on the cation exchange chromatography material employed. Cation exchange chromatography materials with a sulfonic acid group (S) may be used in strong cation exchangers, while cation exchange chromatography materials with a carboxymethyl group (CM) may be used in weak cation exchangers. Strong cation exchangers include, for example SOURCE S, which uses a functional group of methyl sulfate, and SP Sepharose, which uses a functional group of sulfopropyl. Weak cation exchangers include, for example, CM-Cellulose, which uses a functional group of carboxymethyl. SCX may be preferred because a wider range of pH buffers may be used without losing the charge of the strong cation exchanger, allowing for effective separation of analytes with a wide pI range.

Cation exchange chromatography materials are available under different names from a multitude of companies such as, for example, Bio-Rex, Macro-Prep CM (available from BioRad Laboratories, Hercules, Calif., USA), weak cation exchanger WCX 2 (available from Ciphergen, Fremont, Calif., USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare-Amersham Biosciences Europe GmbH, Freiburg, Germany) and sulphonyl immobilized on agarose (e.g. S-Sepharose Fast Flow available from GE Healthcare, Life Sciences, Germany).

Cation exchange chromatography materials include mixed-mode chromatography materials performing a combination of ion exchange and hydrophobic interaction technologies (e.g., Capto adhere, Capto MMC, MEP HyperCell, Eshmuno HCX, etc.), mixed-mode chromatography materials performing a combination of anion exchange and cation exchange technologies (e.g., hydroxyapatite, ceramic hydroxyapatite, etc.), and the like. Cation exchange chromatography materials that may be used in cation exchange chromatography in the present inventions may include, but are not limited to, all the commercially available cation exchange chromatography materials as described above.

While denaturing RPLC-MS is a conventional technique in the characterization of therapeutic proteins, native SCX-MS may provide analytical advantages as described herein. For example, native SCX-MS may provide improved sensitivity and specificity of detection. In cases where the detection limits of RPLC and SCX are comparable, SCX may provide superior data quality and a higher signal-to-noise ratio. SCX may have an improved ability to separate a target analyte from matrix proteins, for example serum proteins in a serum sample, and additionally may have an improved ability to separate biotransformation products of a protein of interest. Exemplary methods for native SCX-MS analysis are described in Yan et al., 2020, J Am Soc Mass Spectrom, 31:2171-2179.

For example, SCX-MS conditions are as follows. The SCX column is YMC BioPro IEX SF 4.6×50 mm, 5 μm. The column temperature is 45° C. Mobile phase A (MPA) comprises 10 mM ammonium acetate, and mobile phase B (MPB) comprises 300 mM ammonium acetate. The flow rate is 0.4 mL/minute. The gradient is: 0-1 minutes: 100% MPA; 1-9 minutes: 100% MPA to 100% MPB; 9-10.5 minutes: 100% MPB; 10.5-10.6 minutes: 100% MPB to 100% MPA; and 10.6-15 minutes: 100% MPA. The MS resolution is set at 12,500 (UHMR). The capillary spray voltage is set at 3.0 kV. The capillary temperature is set at 350° C. The S-lens RF level is set at 200. The in-source fragmentation energy is set at 100. The HCD trapping gas pressure is set at 3. Mass spectra are acquired with an m/z range window between 2000 and 15,000.

The methods of the present inventions include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

The mobile phase used to obtain the eluate from size exclusion chromatography can comprise a volatile salt. The mobile phase can comprise ammonium acetate, ammonium bicarbonate, or ammonium formate, or combinations thereof.

Online coupling of SEC with direct MS detection under near native conditions (native SEC-MS) has gained a lot of interest over the past few years to study mAb HMW species (Rouby et al., supra; Ehkirch A, Hernandez-Alba O, Colas O, Beck A, Guillarme D, Cianferani S. Hyphenation of size exclusion chromatography to native ion mobility mass spectrometry for the analytical characterization of therapeutic antibodies and related products. J Chromatogr B Analyt Technol Biomed Life Sci 2018:1086 (176-183); Haberger M, Leiss M, Heidenreich AK, Pester O, Hafenmair G, Hook M, Bonnington L, Wegele H, Haindl M, Reusch D et al. Rapid characterization of biotherapeutic proteins by size-exclusion chromatography coupled to native mass spectrometry. MAbs 2016:8(2): 331-339. Using MS-compatible mobile phases that can preserve protein conformation and non-covalent interactions, native SEC-MS (nSEC-MS) can provide rapid and improved identification of size variants based on accurate mass measurement.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

LC-MS, such as SCX-MS or SEC-MS, can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. Native MS allows for better spatial resolution compared to non-native MS, improving detection of biotransformation products of a therapeutic protein. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

It is understood that the present inventions are not limited to any of the aforesaid protein(s), antibody(s), monoclonal antibody(s), bispecific antibody(s), protein expression system(s), multisubunit protein(s), protein alkylating agent(s), protein denaturing agent(s), protein reducing agent(s), digestive enzyme(s), hydrolyzing agent(s), sample(s), solid phase substrate(s), capture antibody(s), liquid chromatography system(s), mobile phase(s), mass spectrometer(s), database(s), or bioinformatics tool(s), and any protein(s), antibody(s), monoclonal antibody(s), bispecific antibody(s), protein expression system(s), multisubunit protein(s), protein alkylating agent(s), protein denaturing agent(s), protein reducing agent(s), digestive enzyme(s), hydrolyzing agent(s), sample(s), solid phase substrate(s), capture antibody(s), liquid chromatography system(s), mobile phase(s), mass spectrometer(s), database(s), or bioinformatics tool(s) can be selected by any suitable means.

The present inventions will be more fully understood by reference to the following Examples. They should not, however, be construed as limiting the scope of the invention.

### EXAMPLES

### Example 1. A Native IP and Native LC-MS Workflow for Protein Characterization

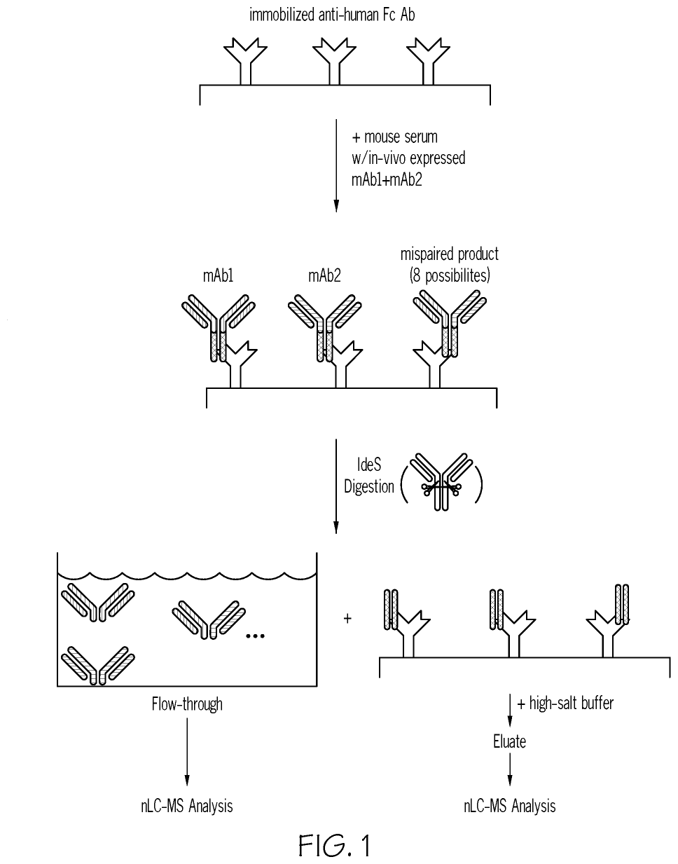

A method for mass spectrometry-based characterization of therapeutic proteins of interest was developed based on native immunoprecipitation (IP) followed by native liquid chromatography-mass spectrometry (nLC-MS). A sample comprising two co-expressed therapeutic monoclonal antibodies (mAbs) is shown in FIG. 1.

Immunoprecipitation was performed on Agilent AssayMap Bravo platform using anti-human Fc antibody for immunocapture. On-cartridge FabRICATOR (IdeS) digestion was used to release the Fab₂ domains from the captured mAbs. Samples were taken from mouse serum with in vivo expression of mAb1 and/or mAb2. Three different in vivo gene delivery platforms in mice were used (platform I, platform II, and platform III).

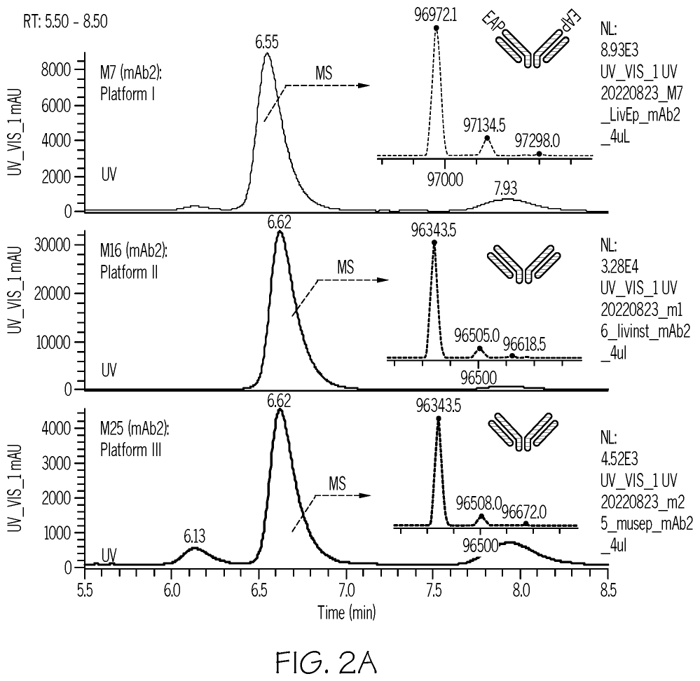

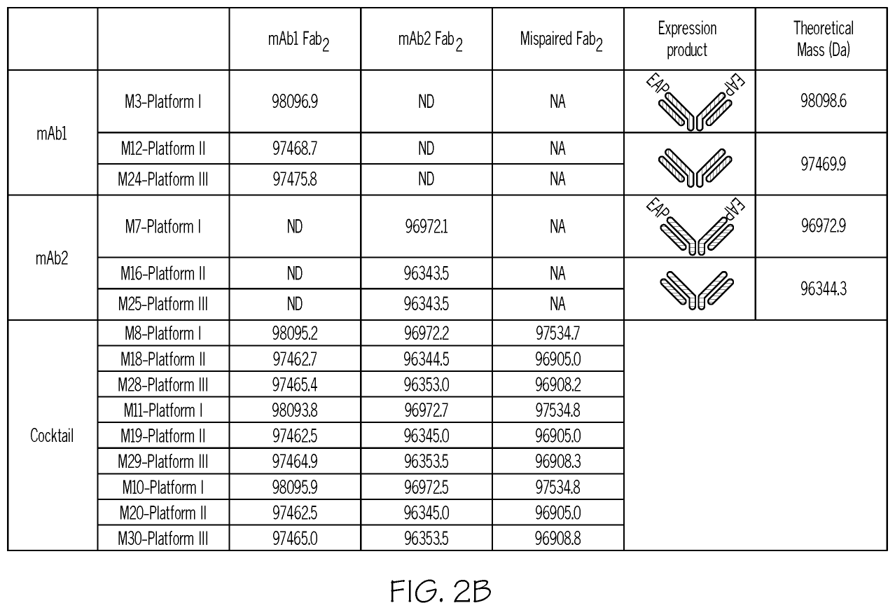

Native size exclusion chromatography-ultraviolet detection/mass spectrometry (nSEC-UV/MS) analysis of Fab₂ fragments eluted from native immunoprecipitation was used to determine changes to the amino acid sequences of mAb1 and mAb2 produced in vivo, as shown in FIG. 2A. An additional “EAP” sequence was detected at the N-terminus of mAbs produced using gene delivery platform I. No additional amino acids were detected in mAbs produced using gene delivery platforms II and III. A summary of measured average masses of Fab₂ in all mouse samples by SEC-MS is shown in FIG. 2B.

This example demonstrates that a protein or proteins of interest in a sample comprising a complex matrix, for example monoclonal antibodies co-expressed in vivo and obtained from serum, can be effectively characterized using a method including native immunoprecipitation, digestion to form subunit fragments, and native size exclusion chromatography-ultraviolet detection/mass spectrometry.

### Example 2. Separation of Mispaired Fab Products Using nSCX-MS

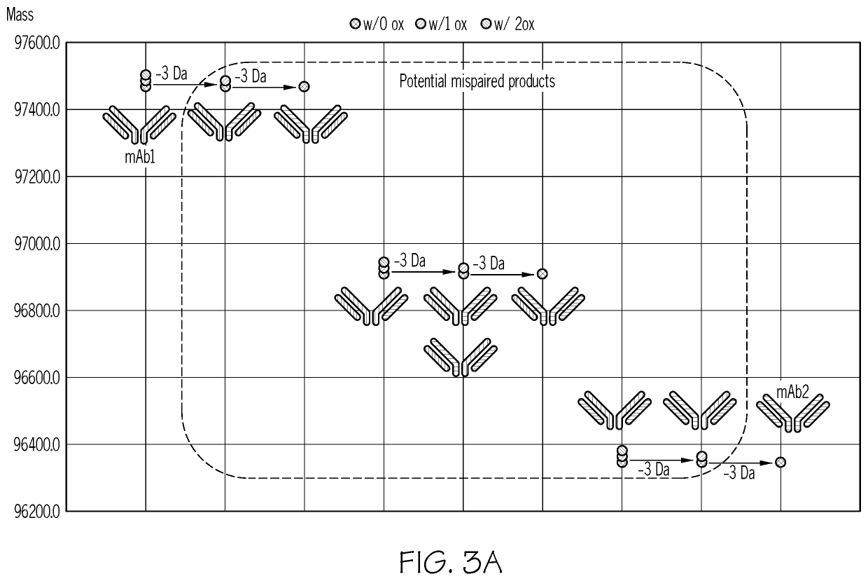

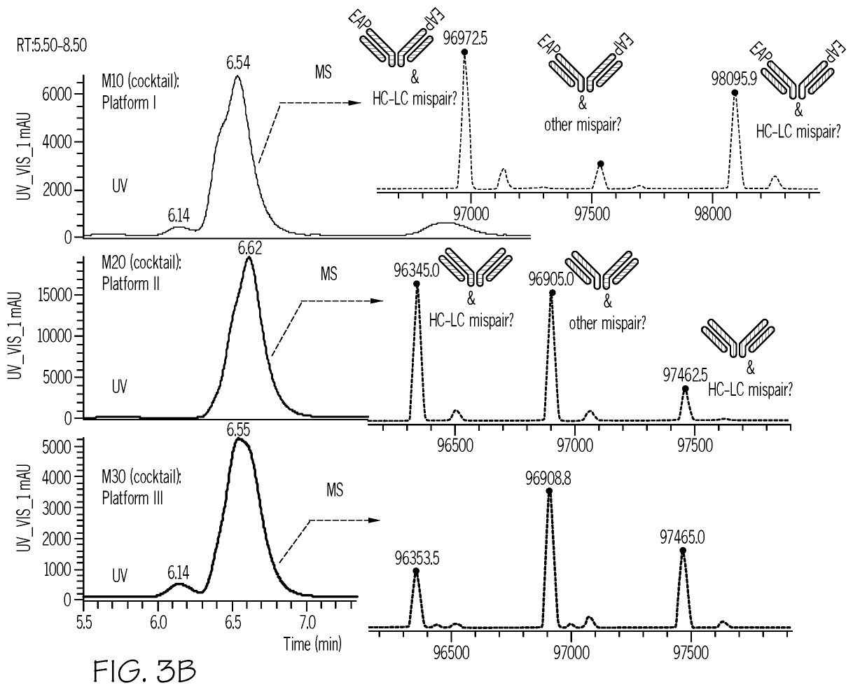

The expression of multisubunit proteins may lead to the production of undesired products, or impurities, comprising mismatched or mispaired subunits. For example, co-expression of two mAbs with different heavy chain (HC) and light chain (LC) sequences can result in the production of up to 10 HC-LC pairs, two of which represent the desired mAbs and eight of which represent mispaired impurities. The theoretical masses of Fab₂ fragments of desired mAb1 and mAb2 products and potential mispaired products is shown in FIG. 3A. The mAb1 and mAb2 heavy chains have a mass difference of 3 Da, and therefore the mispaired Fab₂ products also had similar masses to each other. The effect of oxidation on product mass was also considered because the mAb1 Fd features an oxidation hotspot.

FIG. 3B shows an example of nSEC-UV/MS analysis of co-expressed mAb1 and mAb2 (“cocktail”) Fab₂ fragments from native IP. Mispaired products were identified in all cocktail samples, but could not be confidently differentiated and quantified using a nSEC-MS approach. Due to the small mass difference between mAb1 Fd and mAb2 Fd, the different mispaired products could not be completely differentiated from the correctly paired products, or among themselves, when separating by mass alone.

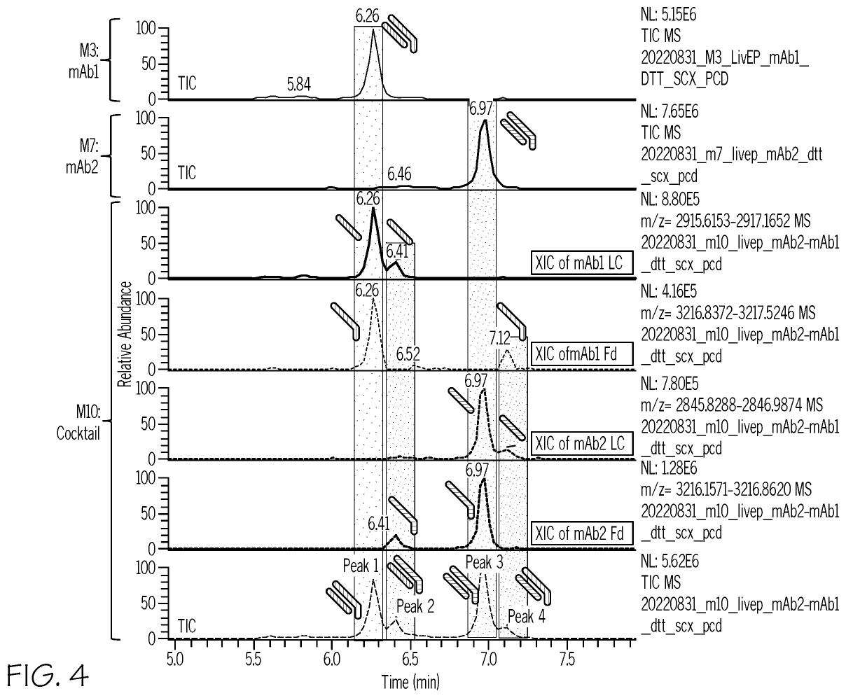

In order to solve this problem, native strong cation exchange chromatography-mass spectrometry (nSCX-MS) was first used to separate Fab fragments. Fab₂ samples eluted from native immunoprecipitation were treated with DTT to produce Fab fragments, and the resulting Fab fragments were subjected to nSCX-MS analysis, as shown in FIG. 4. Different HC-LC pairing products at the Fab level could be confidently separated with nSCX-MS, suggesting that the same method could potentially be useful in separating the differently paired Fab₂ species.

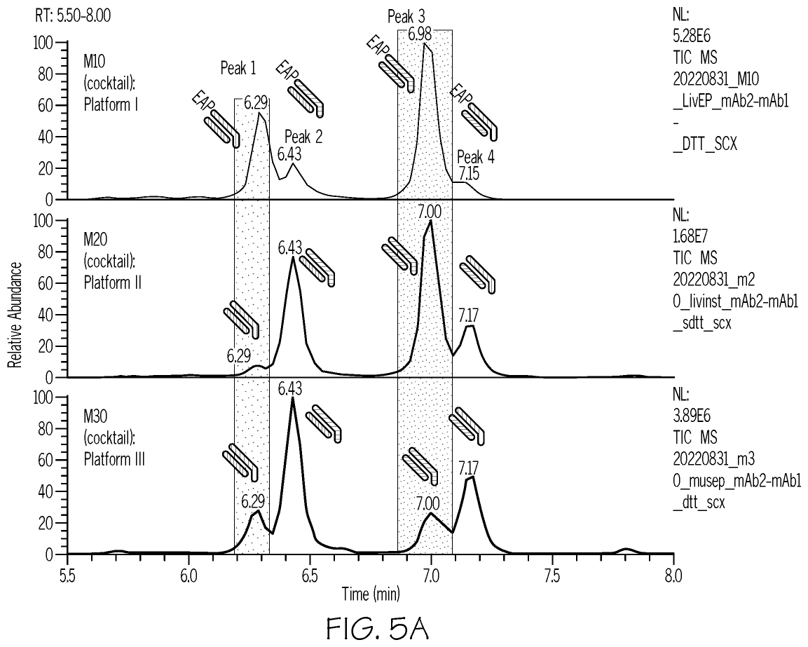

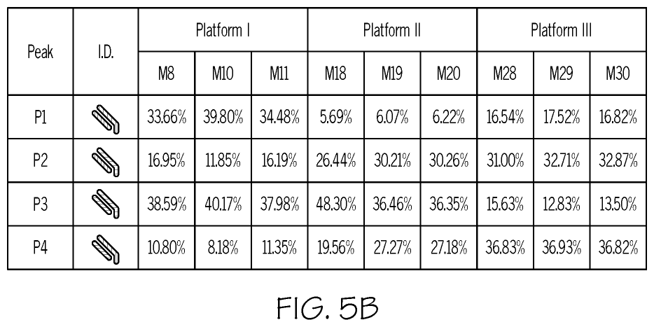

Fab fragments from mAb1 and mAb2 cocktail produced using each of the three gene delivery platforms was analyzed using nSCX-MS, as shown in FIG. 5A and quantified in FIG. 5B. Correct HC-LC pairing for both mAb1 and mAb2 was detected at high abundances in cocktail produced using platform I. Correct mAb1 HC-LC pairing was detected at high abundances in cocktail produced using platform II, but correct mAb2 HC-LC pairing was low using this platform. Both correct mAb1 HC-LC pairing and mAb2 HC-LC pairing were detected at low abundances in cocktail produced using platform III.

This example demonstrates that a protein or proteins of interest in a sample comprising a complex matrix, for example monoclonal antibodies co-expressed in vivo and obtained from serum, can be effectively characterized using a method including native immunoprecipitation, digestion to form subunit fragments, and native strong cation exchange chromatography-mass spectrometry. In particular, nSCX-MS was capable of clearly identifying and quantifying correctly paired and mispaired Fab fragments from an antibody cocktail, and thereby comparing the pairing distribution of antibodies produced using different gene therapy platforms and/or expression platforms.

### Example 3. Separation of Mispaired Fab₂ Products Using nSCX-MS

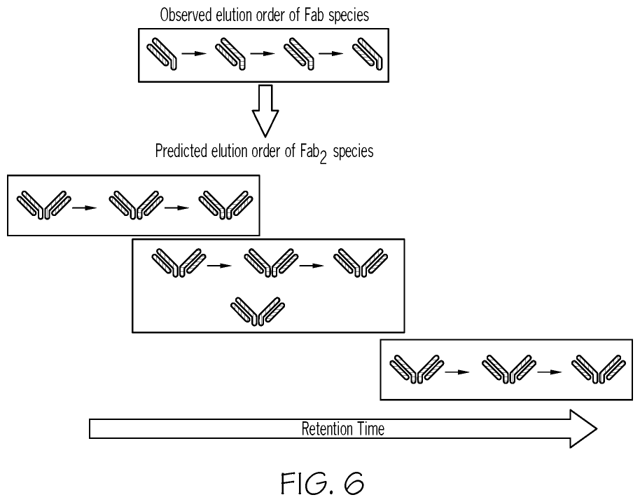

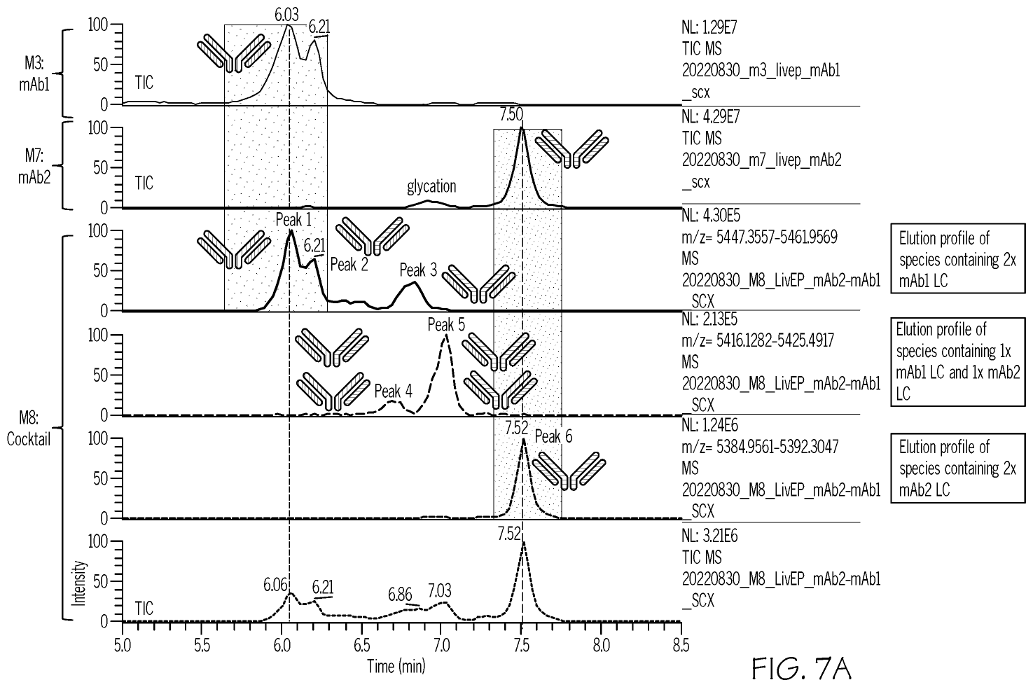

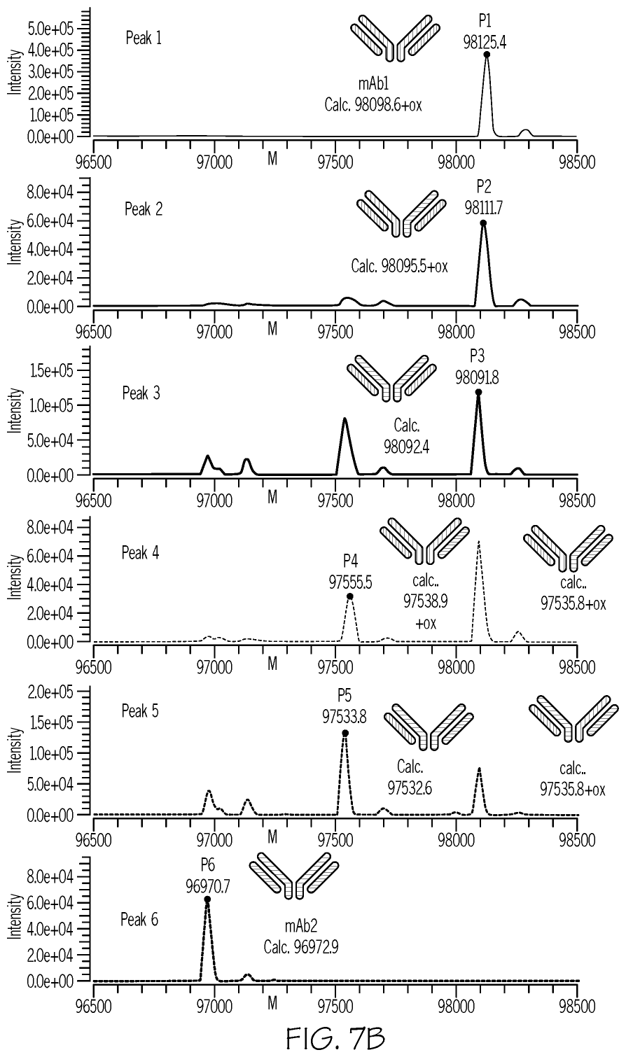

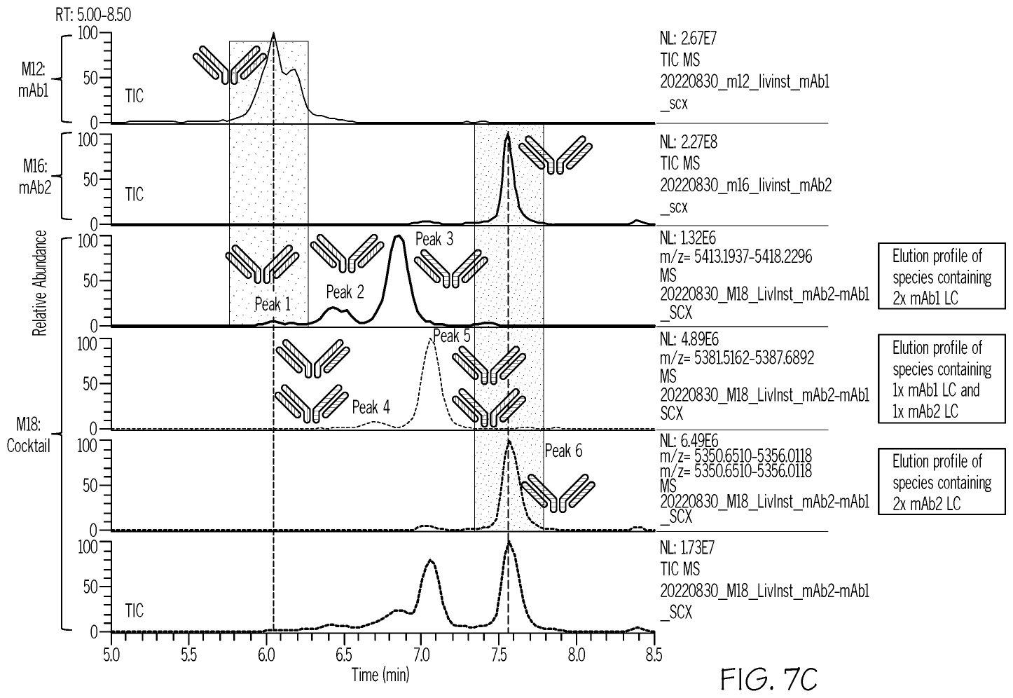

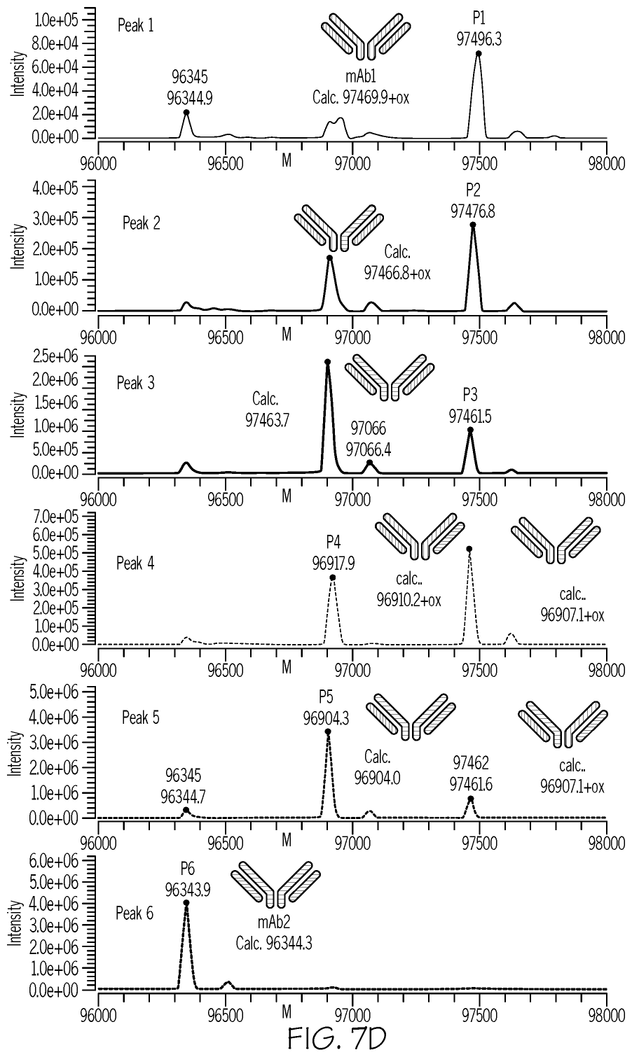

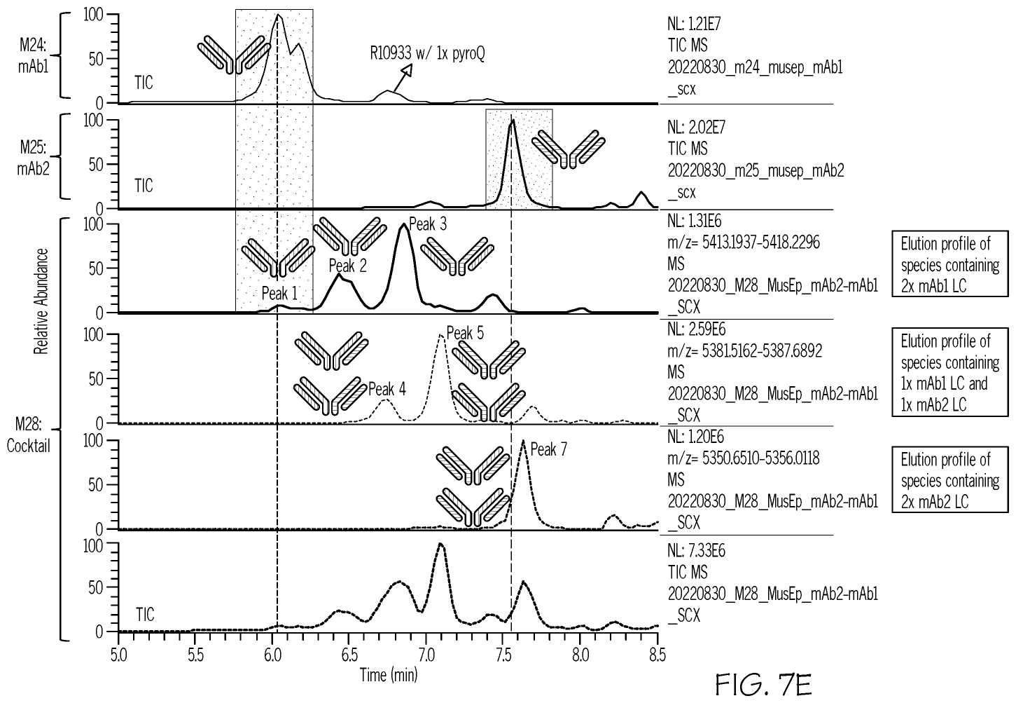

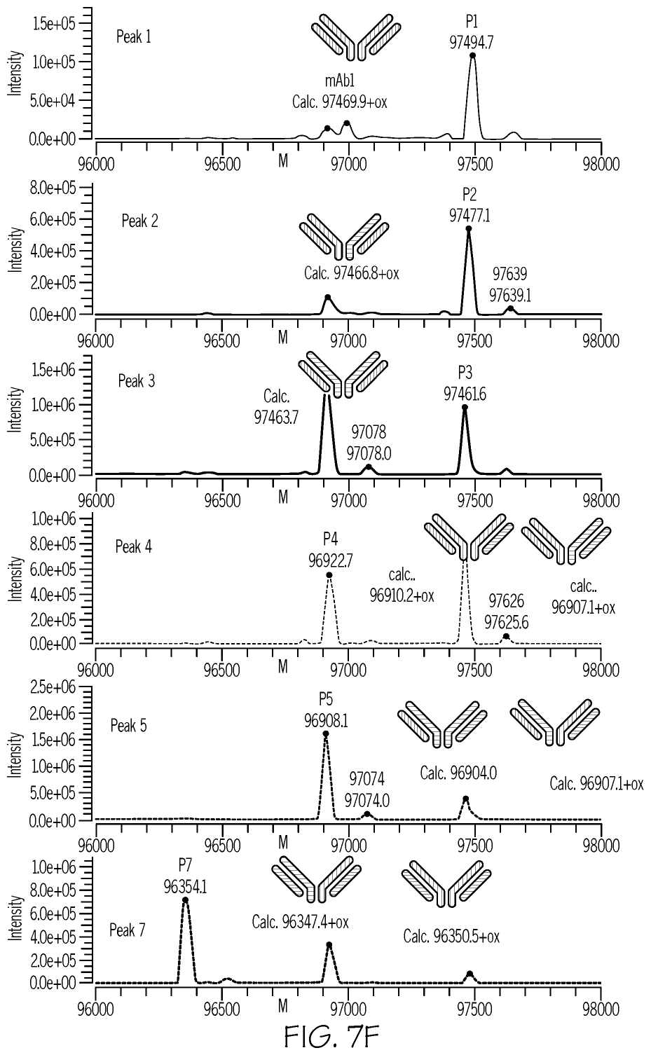

Using the observed elution order of Fab species in nSCX analysis, an elution order of corresponding Fab₂ species was predicted, as illustrated in FIG. 6. Informed by the predicted elution order of each Fab₂ species, nSCX-MS analysis was used to characterize the HC-LC pairing of Fab₂ fragments eluted from native immunoprecipitation of the mAb1 and mAb2 cocktail produced using each of the three gene delivery platforms, as shown in FIGS. 7A-7F. Elution profiles of species containing different combinations of LCs were generated using extracted ion chromatograms (XIC). Based on SCX retention time and mass, correctly paired products could be confidently differentiated from mispaired products.

nSCX-MS analysis of HC-LC pairing of Fab₂ fragments eluted from native immunoprecipitation of the mAb1 and mAb2 cocktail from platform I is shown in FIG. 7A. FIG. 7B shows the corresponding deconvoluted mass spectra. Correctly paired mAb1 and mAb2 were detected at high abundances in cocktail produced using platform I. Mispaired products detected in cocktail produced using platform I could be separated into four groups, including peak 2, 3, 4, and 5, the latter two each containing two possible mispaired products.

nSCX-MS analysis of HC-LC pairing of Fab₂ fragments eluted from native immunoprecipitation of the mAb1 and mAb2 cocktail from platform II is shown in FIG. 7C. FIG. 7D shows the corresponding deconvoluted mass spectra. Correctly paired mAb1 was detected at low abundances in cocktail produced using platform II. Correctly paired mAb2 was detected at high abundances in cocktail produced using platform II. Mispaired products detected in cocktail produced using platform II could be separated into four groups, including peak 2, 3, 4, and 5, the latter two each containing two possible mispaired products.

nSCX-MS analysis of HC-LC pairing of Fab₂ fragments eluted from native immunoprecipitation of the mAb1 and mAb2 cocktail from platform III is shown in FIG. 7E. FIG. 7F shows the corresponding deconvoluted mass spectra. Correctly paired mAb1 was detected at low abundances in cocktail produced using platform III. Correctly paired mAb2 was not detected in cocktail produced using platform III. Mispaired products detected in cocktail produced using platform III could be separated into five groups, including peak 2, 3, 4, 5, and 7, the latter three each containing two possible mispaired products.

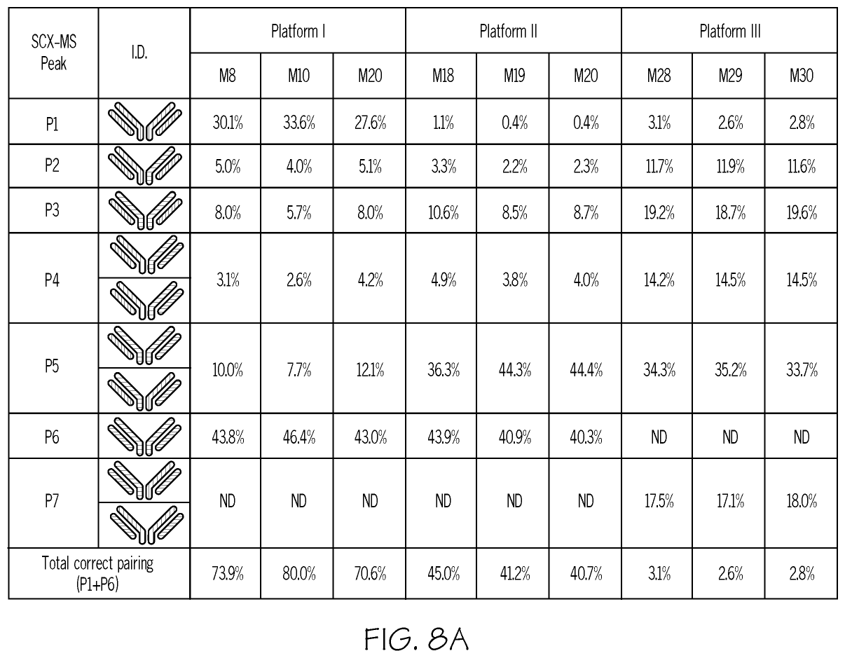

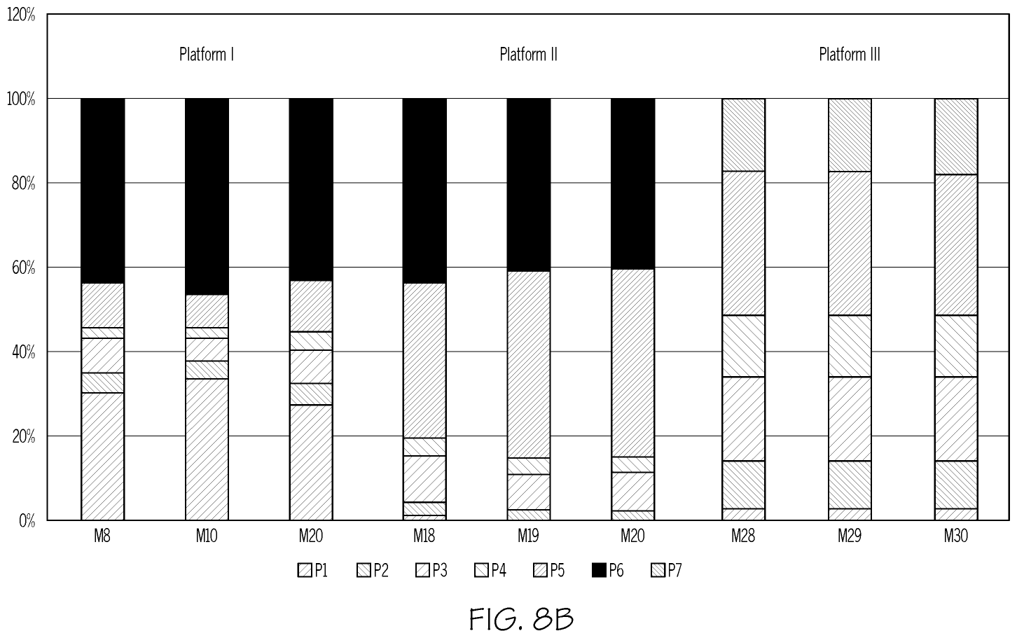

A mass-spectrometry-based quantification of the Fab₂ pairing analysis of a cocktail produced from each platform is shown in FIG. 8A and FIG. 8B. A cocktail produced using platform I contained high levels of both correctly paired mAb1 (˜30%) and correctly paired mAb2 (˜45%). A cocktail produced using platform II contained very low levels of correctly paired mAb1 (<2%) and high levels of correctly paired mAb2 (40-45%). A cocktail produced using platform III contained very low levels of correctly paired mAb1 (<4%), and correctly paired mAb2 was not detected.

This example demonstrates that a protein or proteins of interest in a sample comprising a complex matrix, for example monoclonal antibodies co-expressed in vivo and obtained from serum, can be effectively characterized using a method including native immunoprecipitation, digestion to form subunit fragments, and native strong cation exchange chromatography-mass spectrometry. In particular, nSCX-MS was capable of clearly identifying and quantifying correctly paired and mispaired Fab₂ fragments from an antibody cocktail, and thereby comparing the pairing distribution of antibodies produced using different gene therapy platforms and/or expression platforms. This comparison could be used to optimize and/or select a gene therapy platform and/or expression platform based on its effectiveness at producing correctly paired or assembled protein products or cocktails. Furthermore, this identification and quantification of correctly paired and mispaired protein products could be used to compare two or more protein products or cocktails in order to determine which product or cocktail is more effective at correctly pairing in a given expression system, for example in vitro using a recombinant host cell or in in vivo using gene therapy. Additionally, this identification and quantification could be used to compare the pairing of a given protein product or cocktail using different expression conditions, enrichment conditions, purification conditions, or any other processing step, in order to select or optimize methods and systems for characterizing, analyzing, quantifying, producing, or purifying a protein or proteins of interest.

### Example 4. Glycan Profiling of the Fc Glycan

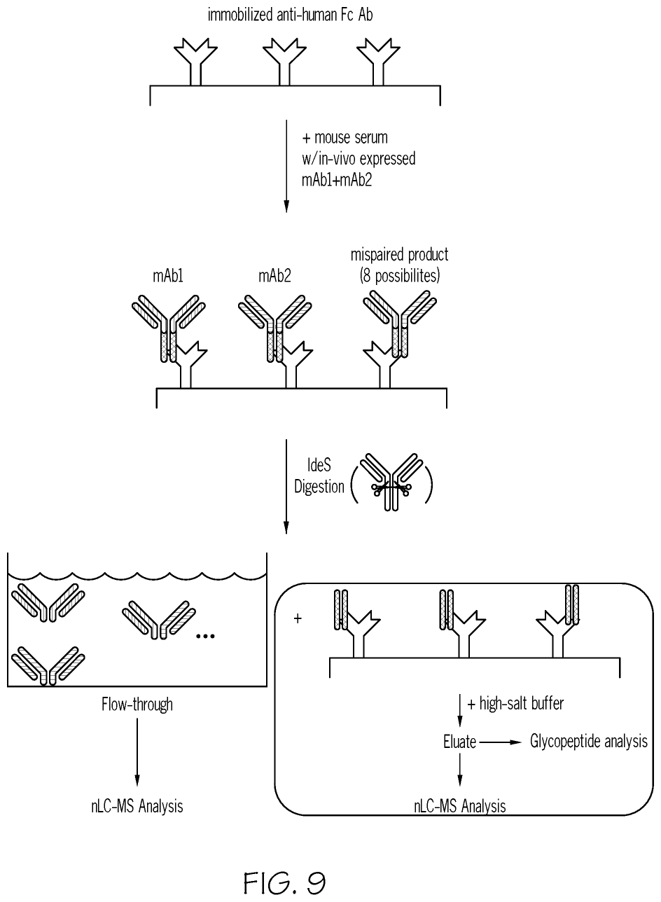

A native IP and nLC-MS workflow was developed for characterizing the glycosylation profile of proteins of interest expressed in vivo. A sample comprising two co-expressed therapeutic mAbs is shown in FIG. 9.

Immunoprecipitation was performed on Agilent AssayMap Bravo platform using anti-human Fc antibody for immunocapture. On-cartridge FabRICATOR (IdeS) digestion was used to release the Fab₂ domains from the captured mAbs. Pierce gentle Ag/Ab elution buffer (pH 6.6) was used for eluting the remaining Fc domain from the cartridge. Samples were taken from mouse serum with in vivo expression of mAb1 and/or mAb2. Three different in vivo gene delivery platforms in mice were used (platform I, platform II, and platform III). Two complementary approaches were used for glycosylation profiling: intact mass analysis and bottom-up analysis.

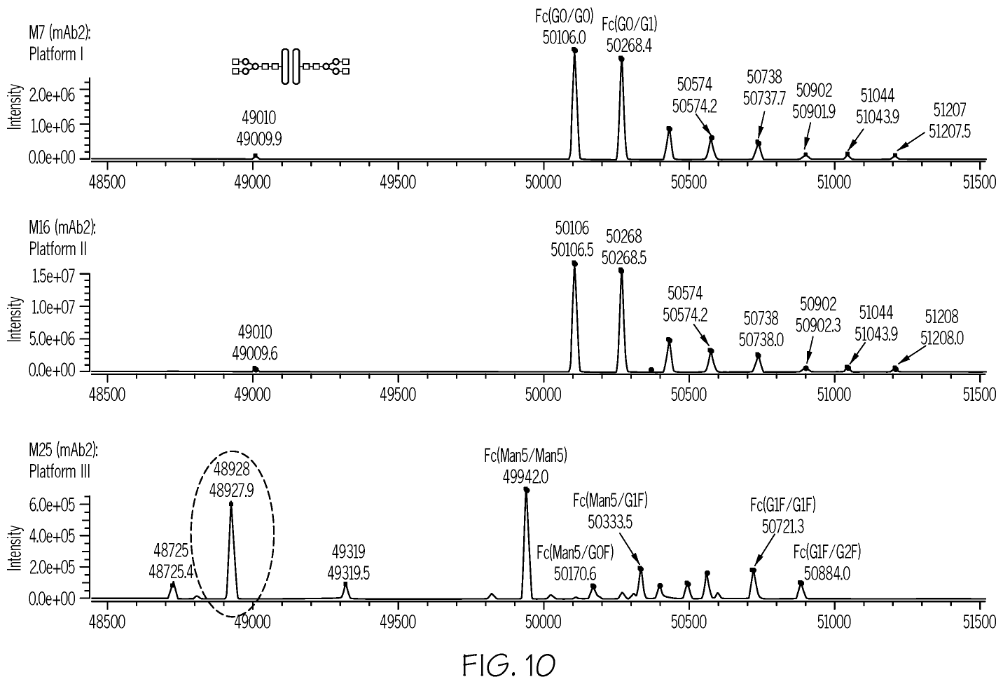

Intact mass analysis of eluted Fc from mAb2 produced using each of the three gene therapy platforms is shown in FIG. 10. nSEC-MS was used for intact mass analysis. Glycoforms identified using intact mass analysis are labeled on the mass spectra. No additional amino acids were found at the C-terminus of the HC in mAbs produced using any of the three platforms. This is consistent with the knowledge that the protein C-terminal K/R amino acids are usually (partially) removed during mammalian cell culture and/or circulation (J. Chrom. A, 705 (1995) 129-134; Biotechnology and Bioengineering, 108 (2011), 2, 401-412).

The major glycoforms found in mAb molecules produced using platform I and platform II included G0 and G1. The major glycoforms found in mAb molecules produced using platform III included Man5, G0F, and G1F.

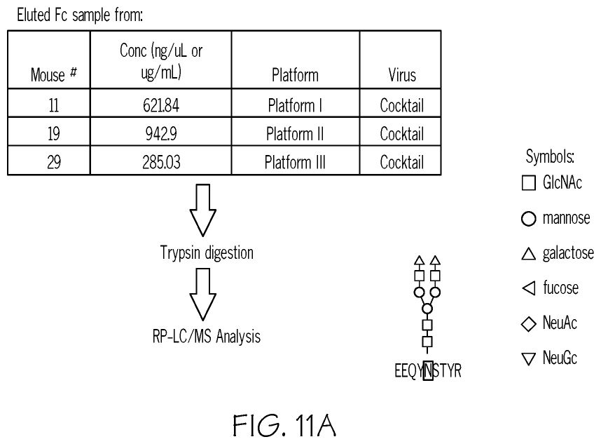

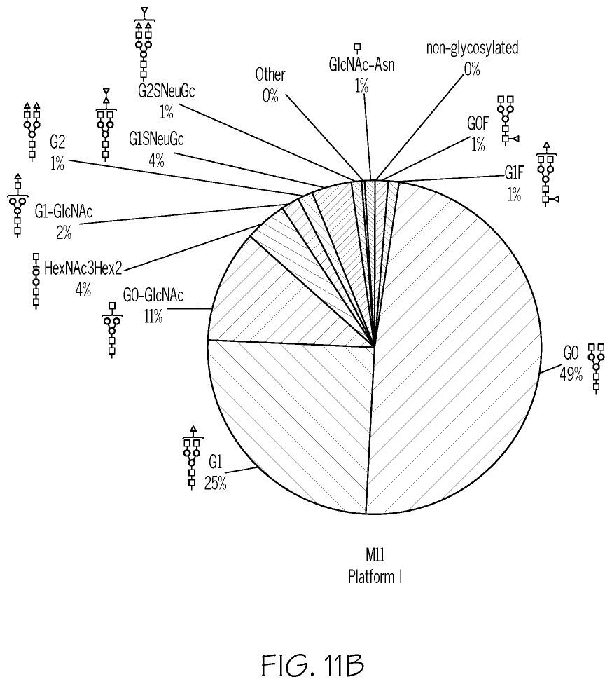

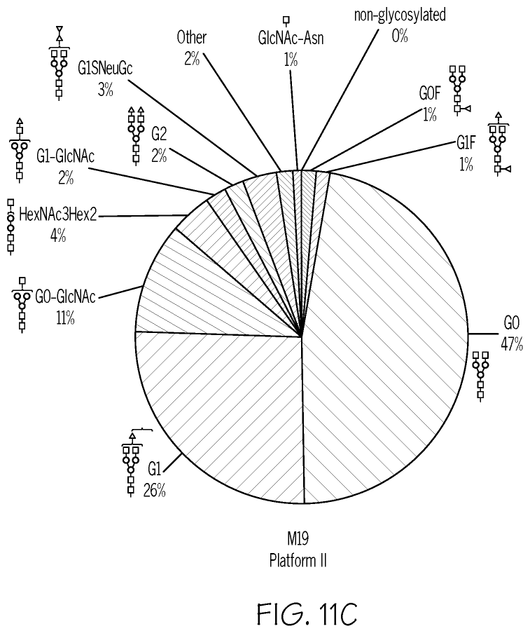

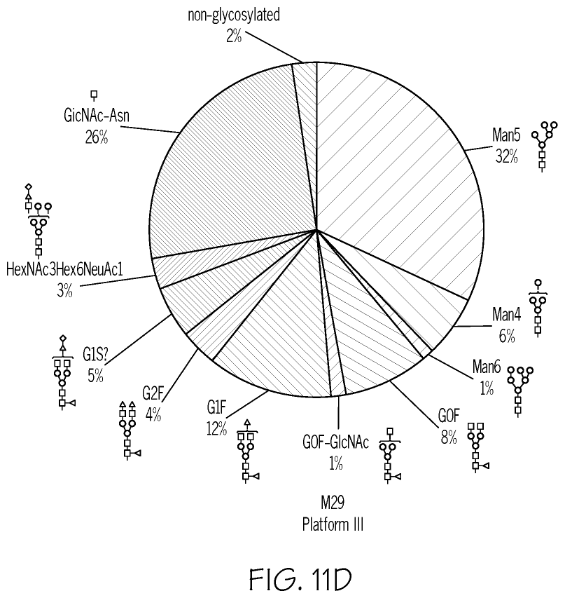

A workflow of a bottom-up analysis of Fc fragments eluted from native immunoprecipitation of mAb1 and mAb2 cocktail is shown in FIG. 11A. Eluted Fc fragments were subjected to trypsin digestion to form a peptide digest, followed by reversed phase (RP)-LC/MS analysis. The peptide containing the N-glycosylation site of the Fc fragment is shown. The results of a bottom-up analysis of Fc glycosylation for cocktail produced from platform I, platform II, and platform III are shown in FIG. 11B, FIG. 11C and FIG. 11D respectively. Both samples expressed from platform I and platform II had >99% occupancy at the Fc glycan site (EEQYN300STYR). G0 and G1 were the major glycans detected. Samples expressed using platform III had ˜72% “normal” glycan occupancy (including mainly Man5, Man4, G0F, and G1F) and ˜26% of GlcNAc occupancy on the same Fc Asn site. It should be noted that G0-GlcNAc could be the result of an in-source fragmentation product of G0, and Man4 could be the result of an in-source fragmentation product of Man5.

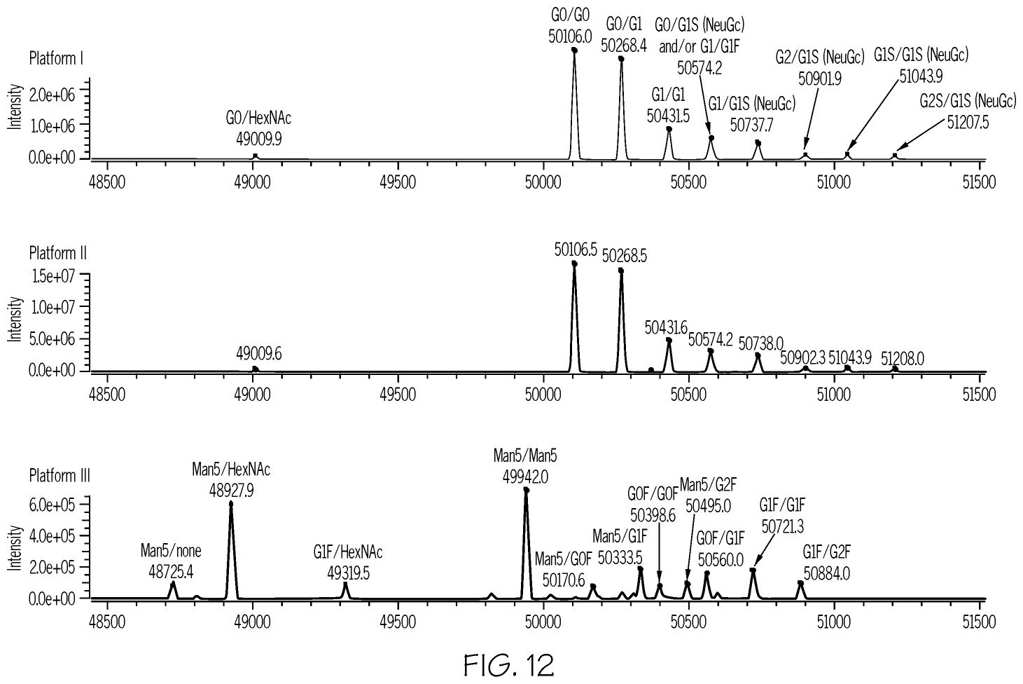

The glycoforms identified using intact mass analysis and bottom-up analysis are compared in FIG. 12, with results unique to bottom-up analysis shown in red. The intact mass analysis used a mAb2 sample and not a cocktail sample, but because the Fc fragment is identical between mAb1 and mAb2, a direct comparison can still be made between a mAb2 sample and a cocktail sample. A comparison shows that bottom-up glycopeptide analysis resulted in more consistent and more detailed Fc glycan profiling compared to intact mass analysis.

This example demonstrates that a protein or proteins of interest in a sample comprising a complex matrix, for example monoclonal antibodies co-expressed in vivo and obtained from serum, can be effectively characterized using a method including native immunoprecipitation, digestion to form subunit fragments, and intact mass analysis and/or bottom-up analysis. In particular, using tryptic digestion and RP-LC/MS analysis of eluted Fc fragments, glycosylation profiles of antibodies from antibody cocktails produced using three different gene therapy platforms could be obtained. These glycosylation profiles could be used, for example, to compare gene therapy platforms and/or expression platforms, to compare proteins of interest, and/or to compare methods of production or purification, in order to select an optimal glycosylation profile for proteins of interest.

1. A method for characterizing an assembly of subunits of at least one multisubunit protein of interest, comprising:
(a) contacting a sample including the at least one multisubunit protein of interest to a solid-phase substrate under native or near-native conditions, wherein the solid-phase substrate comprises a capture antibody that binds to at least one subunit of the at least one multisubunit protein of interest, to form immobilized proteins;
(b) eluting the immobilized proteins to form an enriched sample; and
(c) subjecting the enriched sample to liquid chromatography-mass spectrometry (LC-MS) analysis under native conditions to characterize the assembly of subunits of the at least one multisubunit protein of interest.

2. The method of claim 1, wherein the at least one multisubunit protein is selected from a group consisting of an antibody, a monoclonal antibody, a bispecific antibody, an antibody fragment, an antibody-derived protein, an antigen-binding protein, an antibody-drug conjugate, or a fusion protein.

3. The method of claim 1, wherein the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

4. A method for identifying and/or quantifying correctly paired antibodies from a sample comprising two or more antibodies co-expressed in vivo, comprising:
(a) contacting a sample including two or more antibodies co-expressed in vivo to a solid-phase substrate under native or near-native conditions to form immobilized antibodies;
(b) contacting the immobilized antibodies to a digestive enzyme to produce unbound fragments of the antibodies;
(c) eluting the unbound fragments; and
(d) subjecting he eluted fragments to native strong cation exchange chromatography-mass spectrometry (nSCX-MS) analysis to identify and/or quantify correctly paired antibodies.

5. The method of claim 4, wherein the solid-phase substrate is selected from a group consisting of a microplate, resin, and beads.

6. The method of claim 4, wherein the solid-phase substrate comprises beads.

7. The method of claim 6, wherein the beads are agarose beads or magnetic beads.

8. The method of claim 4, wherein the binding is performed by an antibody-binding molecule adhered to the solid-phase substrate.

9. The method of claim 8, wherein the antibody-binding molecule is Protein A, Protein G, or an anti-Fc antibody.

10. The method of claim 4, wherein the digestive enzyme is selected from a group consisting of pepsin, trypsin, Tryp-N, chymotrypsin, Lys-N, Lys-C, Asp-N, Arg-C, Glu-C, papain, IdeS, or a variant thereof.

11. The method of claim 4, wherein the unbound fragments are Fab fragments, Fab′ fragments, Fab₂ fragments, F(ab′)₂ fragments, Fc fragments, Fv fragments, Fd fragments, or Fd′ fragments.

12-24. (canceled)

25. A method for characterizing at least one antibody of interest expressed in vivo, comprising:
(a) contacting a sample including at least one antibody of interest expressed in vivo to a solid-phase substrate under native or near-native conditions, wherein the solid-phase substrate comprises anti-Fc antibodies, to form immobilized antibodies;
(b) contacting the immobilized antibodies to digestive conditions including IdeS or a variant thereof to form free Fab₂ fragments;
(c) eluting the free Fab₂ fragments to form eluted Fab₂ fragments; and
(d) subjecting the eluted Fab₂ fragments to native size exclusion chromatography-mass spectrometry (nSEC-MS) or native strong cation exchange chromatography-mass spectrometry (nSCX-MS) analysis to characterize the at least one antibody of interest.

26-30. (canceled)

31. A method for characterizing a glycosylation profile of a protein of interest, comprising:
(a) contacting a sample including a protein of interest to a solid-phase substrate under native or near-native conditions, wherein the solid-phase substrate comprises a capture antibody that binds to the protein of interest, to form immobilized proteins;
(b) eluting the immobilized proteins to form an enriched sample; and
(c) subjecting the enriched sample to native liquid chromatography-mass spectrometry analysis to characterize a glycosylation profile of the protein of interest.

32-37. (canceled)

38. The method according to claim 31, wherein the protein of interest is an antibody.

39. The method according to claim 38, wherein the sample of step (a) comprises at least two antibodies.

40. The method according to claim 38, wherein the solid-phase substrate comprises anti-Fc antibodies to immobilize antibodies.

41. The method according to claim 40, wherein antibodies immobilized to the solid-phase substrate are subjected to digestive conditions including IdeS or a variant thereof to form immobilized Fc fragments.

42. The method according to claim 41, wherein the enriched sample comprises eluted Fc fragments.

43. The method according to claim 42, wherein the eluted Fc fragments are digested to form a peptide digest.

44. The method according to claim 43, wherein the peptide digest is subjected to reversed phase liquid chromatography-mass spectrometry (RPLC-MS) analysis to characterize glycosylation profiles of the antibodies.
